(19) United States
(12) Patent Application Publication
(10) Pub. No.: US 2009/0264640 A1
(43) Pub. Date: Oct. 22, 2009

(54) Recovery of dimethylformamide and other solvents from process streams of manufacture of trichlorogalactosucrose

(75) Inventors: Rakesh Ratnam, Karnataka (IN); Sundeep Aurora, Karnataka (IN); n/a Subramaniyan, Karnataka (IN)

Correspondence Address: THE NATH LAW GROUP, 112 South West Street, Alexandria, VA, 22314, US

(73) Assignee: V.B. MEDICARE PVT. LTD, KARNATAKA (IN)

(21) Appl. No.: 12/227,595

(22) PCT Filed: May 16, 2007
(86) PCT No.: PCT/IN2007/000197

§ 371 Date: May 1, 2009

(30) Foreign Application Priority Data
May 23, 2006 (IN) 779/MUM/2006

(51) Int. Cl. C07H 3/04; C07C 231/24

(52) U.S. Cl. 536/123.13; 564/497

(57) ABSTRACT

This invention comprises an improved process for recovery and purification of DMF from an aqueous process stream containing DMF with or without inorganic impurities, particularly from process stream of a process of manufacture of the high intensity sweetener Trichlorogalactosucrose, by adsorption on an adsorbent having selective affinity towards dimethylformamide, followed by elution in pure form by eluting by an appropriate eluent, including methanol.

### TECHNICAL FIELD

The present invention relates to methods of recovery of N—N-dimethylformamide from process streams of production of Trichlorogalactosucrose, i.e. 1′-6′-Dichloro-1′-6′-DIDEOXY-β-Fructofuranasyl-4-chloro-4-deoxy-galactopyranoside (TGS).

### BACKGROUND OF INVENTION

The most economical way of recovery of DMF from the Process Streams of TGS manufacture is described wherein, the tertiary amide is adsorbed on to an Affinity chromatographic resin. The other impurities are washed away and pure DMF was eluted using suitable solvents.

The manufacture of TGS involves the protection of the 6^{th} primary position of sucrose. This is done by first dissolving sucrose in a suitable solvent. The preferable solvent is a tertiary amide such as N—N-dimethylformamide (DMF), Dimethyl acetamide, etc. Further after the formation of the suitable 6-O-protected ester of sucrose, the chlorination is carried out using a Vilsmeier-Haack reagent (Vilsmeier reagent). This Vilsmeier reagent is generated by reacting a chlorinating reagent such as Thionyl chloride, Phosphorus oxychloride, Phosphorus pentachloride, etc with a tertiary amide such as N—N, Dimethylformamide, etc. The reaction is carried out with excess of DMF, so that DMF itself acts as a medium for carrying out the chlorination reaction.

The chlorination reaction forms TGS, the artificial sweetener, along with various other chlorinated sugar derivatives as impurities. The solvent, DMF from the reaction mixture during the isolation of the TGS, has to be recovered. DMF is a substantial cost factor in the process costing for the manufacture of TGS. The economical way of solvent recovery forms a part of process design, wherein the recovered solvent is free from impurities and can be re-used further for subsequent batch cycle. This is also necessary to avoid problem of handling of DMF in effluents from the point of pollution control.

However, high boiling point and decomposition when heated above 80-100° C. are the properties of DMF or any tertiary amide, which make a recovery of DMF difficult in conventional distillation systems.

When DMF is distilled off at lower temperatures under vacuum or distilled at higher temperatures, the energy cost associated with it is enormous. So it is impractical to recover DMF in an economical way by the process of conventional distillation.

It is an object of this invention to find out more efficient and more convenient methods of recovery of DMF from process streams.

### PRIOR ART

Navia et al (1996^{a}) in U.S. Pat. No. 5,530,106 and Navia et al (1996^{b}) in U.S. Pat. No. 5,498,709 recovered DMF from other constituents of process stream of manufacture of TGS by steam stripping. However, this does not lead to total removal of DMF on one hand, leads to large increase in volume of reactants left behind in the process stream and further, the removed DMF needs to be again recovered further.

Removal of DMF has also been achieved by Ratnam et al in a patent application no. PCT/IN2004/000142 by drying under mild conditions, including use of Agitated Thin Film Dryer. However, this process recovers DMF as an aqueous solution from which its recovery in pure form again involves distillation at a higher temperature, which involves loss of this precious solvent. An improved method based on azeotropic distillation is subject matter of another patent application of inventors of this application which involves repeated distillations until about 5% DMF is left behind in the process flow; however, this involves repeated distillations and the DMF from the azeotrope needs to be recovered by a further process.

A simpler process that can be completed in minimum number of steps and achieving recovery of DMF in pure form is highly desirable.

### SUMMARY OF INVENTION

The process of this invention achieves isolation of a tertiary amide, particularly DMF from other aqueous and inorganic constituents of a process flow by selective adsorption of a tertiary amide on an adsorbent. The constituents that do not get adsorbed are washed away and the tertiary amide desorbed from the said adsorbent by a non-aqueous eluent solvent that can be removed from the eluted out mixture by distillation under atmospheric or reduced pressure.

One preferred embodiment a process stream to which this invention can be applied for recovery of a tertiary amide comprises recovery of DMF from the process streams of TGS manufacture wherein DMF is adsorbed on to a bed of a resin in a chromatography column, impurities are washed away and pure DMF is eluted using suitable solvents.

The said affinity chromatographic resins are with groups on them capable of adsorbing an organic solvent including DMF selectively/preferentially over aqueous and/or inorganic constituents, and comprise subsequent elution and recovery of the adsorbed solvent in pure form by using an appropriate eluent. Here direct energy cost of solvent recovery is dramatically reduced and the quality of the solvent recovered is also higher in purity. A resin HP20 from Diaion (Mitsubishi Chemical Corporation, 33-8 Shiba 5-chome, Minato-ku, Tokyo 108-0014 Japan) is an illustrative chromatographic resin disclosed here that has selective affinity towards a tertiary amide, particularly towards DMF, in preference to aqueous and/or inorganic constituents of a process stream.

This invention may also be used for recovery of a tertiary amide from a process flow of any other organic synthesis reaction by applying affinity chromatography as embodiments of this invention. For example: in the synthesis of Roxythromycin antibiotic from erythromycin, DMF is used as a solvent and here in this process also DMF can be recovered by resin based chromatographic process

### DETAILED DESCRIPTION OF INVENTION

Throughout this specification, mention of a singular, unless the context does not permit, also includes its plural. Mention of a reactant or a reaction condition is not to be construed to limit the claims but is to be construed to be only to illustrate a most preferred embodiment of the invention with respect to that factor and any other alternative performing the same function and that can be used as an alternative within the scope of the claims are to be construed as being covered by that disclosure. Thus a mention of “a tertiary amide” includes any and every tertiary amide or tertiary amides; mention of “DMF” includes any of other tertiary amides including dimethyl acetamide, N-methylpyrrolidine and the like that can perform the same function when used in place of DMF and mention of “an affinity chromatographic resin” includes all types of chromatographic resins that can adsorb a chemical in preference to other chemical constituents of a process stream in the in the described context, here a tertiary amide in preference to an aqueous and/or inorganic constituent of a process flow, in addition to the preferred and specified affinity chromatography resin in the specification.

An embodiment of this invention comprises recovery of a tertiary amide, preferably DMF, from a process flow obtained in a process of manufacture of DMF that comprises DMF, water and inorganic salts by selective adsorption on an adsorbent.

One embodiment of this invention, thus, comprises identification of an adsorbent as an affinity chromatography resin capable of selective adsorption of DMF, the preferred tertiary amide, from process streams. Preferred embodiment of process of adsorption is chromatography on a column packed with the preferred adsorbent.

In one preferred embodiment of the process, the process stream from the TGS manufacture containing DMF is directly passed on through a chromatographic resin packed in a Stainless Steel (SS) column. The DMF process stream is passed at a particular flow rate as per the design considerations. The DMF selectively gets adsorbed to the resin and the other impurities with water pass through the outlet of the column. The resin is then washed to remove any adhering impurities. The DMF adsorbed in the resin is eluted out by suitable solvents such as methanol, acetone, etc. The DMF solvent mixture is then subjected to low temperature distillation and the pure DMF is recovered.

The embodiments of resins used for affinity chromatography of this invention are aromatically engineered synthetic adsorbents. The base synthetic material is styrene coupled with divinyl benzene. These specially cross linked resins are highly porous and can hold large molecules in it and can also be eluted out easily. These resins are used for recovery or purification of variety of solvents. Attaching to these resins functional groups, which have selective or preferential affinity towards the molecule of interest, here a tertiary amide, serves the purpose of making them useful for selective adsorption and purification applications.

The particular embodiment of an adsorbent useful for practicing this invention is illustrated by HP20 resin obtained from Diaion (Mitsubishi Chemical Corporation, 33-8 Shiba 5-chome, Minato-ku, Tokyo 108-0014 Japan). The HP20 resin is a standard grade of Aromatic type adsorbent based on crosslinked polystyrenic matrix used in different industrial fields including extraction of antibiotic intermediates from fermentation broth, separation of peptides or food additives, debittering of citrus juice etc. The HP20 resin is a polystyrene base coupled with benzene ring, which makes it highly hydrophobic.

This invention may also be used for recovery of a tertiary amide from a process flow of any other organic synthesis reaction by applying affinity chromatography as embodiments of this invention. For example: in the synthesis of Roxythromycin antibiotic from erythromycin, DMF is used as a solvent and here in this process also DMF can be recovered by resin based chromatographic process

The process stream from the TGS manufacture could be DMF in any one of the following mixtures

a) DMF in aqueous solution
b) DMF in aqueous solution along with inorganic salts

The embodiments of a process stream containing DMF on which process of this invention can be adapted for DMF recovery comprises aqueous mixtures of DMF obtained as a first step of recovery from a reaction mixture generated in one or more of a process of TGS manufacture described by U.S. Pat. Nos. 4,801,700, 4,826,962, 4,889,928, 4,980,463, 5,023,329, 5,089,608, 5,498,709 and 5,530,106. This list is illustrative and not claimed to be exhaustive or limiting. Many more embodiments of process streams can be considered for adaptation of this invention for recovery of DMF and all these are considered to be included in this disclosure.

After recovery of DMF in this invention in the form of a mixture eluted from affinity chromatographic column, usually the amount of DMF in the preferred eluent methanol is about 40-50%. Recovery of DMF from this mixture/solution is easier, more convenient and less energy expensive than DMF recovery from a DMF:water mixture usually obtained in conventional prior art processes cited above wherein DMF content in the aqueous mixture is usually not more than 15-18%. This DMF:water mixture, if subjected to atmospheric distillation, the temperature should be 100° C. and DMF slowly decomposes at this temperature. Also some percent of DMF and water will form azeotrope and result in a water DMF mixture containing about 80-85% of DMF in water. This needs to be again rectified in a distillation column to obtain 95% and above of DMF content for satisfactory recovery. Distillation at lower pressure to remove water is not as economical as compared to removal of methanol. The boiling point difference between methanol and DMF is very high and they do not form any azeotrope, whereas DMF and water will have to go through two distillation steps to recover DMF in high percentage and the energy cost of these operations become prohibitive compared to the price of the DMF recovered.

Same approach shall cover a tertiary amide, which can be used in alternative to DMF in a reaction such as Dimethyl acetamide used in Vilsmeier reagent preparation.

### Example 1

### DMF Recovery from a Process Stream from Chlorination of Sucrose-6-Ester Using Chlorination of Sucrose-6-Ester Using Vilsmeier Generated from Thionyl Chloride and DMF

475 L of DMF was taken in a GLR and 16 kg of charcoal was added to it and stirred. Nitrogen sparging into the reaction mass was started and 344 L of thionyl chloride was added dropwise controlling the temperature between 40 and 45° C. and with constant stirring. After the completion of addition of thionyl chloride, the mass was stirred at 45° C. for 60 minutes and then cooled to 0-5° C. 80 kg of 88% sucrose-6-acetate in DMF was added to the mass slowly and the temperature was controlled below 5° C. Then the mass was allowed to come to ambient temperature (30-35° C.) and was stirred for 3 hours. Then the mass was heated to 85° C. and maintained for 60 minutes, again heated to 100° C., maintained for 6 hours and further heated to 114° C. and maintained for 90 minutes. Then the chlorinated mass was neutralized using 7% Ammonia solution in a continuous quenching system up to pH 7.0

The neutralized mass volume was found to be 3500 L and the DMF content was 18%. It also contained Chlorinated sucrose derivatives and inorganic salts dissolved in it.

### Example 2

### DMF Recovery from Aqueous Process Stream Containing Inorganic Salts Generated by Effluents from a Process of Affinity Chromatographic Separation of TGS and Related Compounds

Generation of the process stream: 3000 L of a process stream from TGS manufacture from Example 1 containing 18% of DMF and dissolved inorganic salts from chlorination was taken for DMF recovery.

The solution was passed through ADS 600 resin obtained from Thermax packed in SS column. The flow through from the column had DMF, inorganic salts and water and the 6-acetyl TGS was bound to the resin column. The column was then washed with water to remove any DMF and inorganics adhering to the resin. Then the flow through and washings collected was taken for DMF recovery. The total volume was 3500 L containing 15.7% DMF.

Recovery of DMF by affinity chromatography: 800 L of collected flow through solution was passed through 1200 L of HP20 resin obtained from Diaion (Mitsubishi Chemical Corporation, 33-8 Shiba 5-chome, Minato-ku, Tokyo 108-0014 Japan) packed in SS column. The solution was passed at a flow rate of 450 L/H. The flow through from the column had inorganic salts in water. The DMF was selectively adsorbed based on hydrophobic interaction chromatography to the resin. This flow through stream was collected and taken for waste management.

After the solution was passed, the column was washed with 2400 L of DM water at 450 L/H. Then the adsorbed DMF in the resin was eluted with 1500 L of methanol.

The DMF along with methanol was collected from the bottom of the column and was subjected to distillation at 45° C. under vacuum for methanol removal. The DMF obtained was checked for purity by GC and was found to be 97.8%. The overall yield of DMF from the recovery stream was 95%.

### Example 3

### DMF Recovery from Aqueous Process Stream Containing Inorganic Salts Generated by Drying by Agitated Thin Film Dryer in a Process of Manufacture of TGS

Generation of the process stream: 500 L of neutralized mass from Example 1 was passed through the Agitated Thin Film Dryer where the mass was dried under vacuum and the temperature was maintained below 45° C. The solids obtained was a mixture of inorganic salts and chlorinated sucrose derivatives including 6-acetyl TGS. This solids were taken for extraction and isolation of TGS by suitable methods.

The solvents that were removed from the feed stream to ATFD were condensed through a high efficiency condensation system where the DMF solution in water was obtained. This solution had 16% of DMF and was taken for DMF recovery.

Recovery of DMF by affinity chromatography: This solution was passed through 550 L of HP20 (details as in Example 2) packed in SS column. The solution was passed at a flow rate of 175 L/H. The flow through from the column was water and was sent directly to waste management. This stream was collected and taken for waste management. The DMF was selectively adsorbed to the resin. After the solution was passed, the column was washed with 1200 L of DM water at 175 L/H. Then the adsorbed DMF in the resin was eluted with 550 L of methanol.

The DMF along with methanol was collected from the bottom of the column and was subjected to distillation at 45° C. under vacuum for methanol removal. The DMF obtained was checked for purity by GC and was found to be 96.2%. The overall yield of DMF from the recovery stream was 94%.

### Example 4

### Dimethyl Acetamide Recovery from Chlorination of Sucrose-6-Ester Using Vilsmeier Generated from Thionyl Chloride and Dimethyl Acetamide

Generation of the process stream: 4.85 L of Dimethyl acetamide was taken in a GLR and 0.18 kg of charcoal was added to it and stirred. Nitrogen sparging into the reaction mass was started and 3.44 L of thionyl chloride was added dropwise controlling the temperature between 40 and 45° C. and with constant stirring. After the completion of addition of thionyl chloride, the mass was stirred at 45° C. for 60 minutes and then cooled to 0-5° C. 0.8 kg of 82% sucrose-6-acetate in Dimethylacetamide was added to the mass slowly and the temperature was controlled below 5° C.

Then the mass was allowed to ambient temperature and was stirred for 3 hours. Then the mass was heated to 85° C. and maintained for 60 minutes, again heated to 100° C., maintained for 6 hours and further heated to 114° C. and maintained for 90 minutes. Then the chlorinated mass was neutralized using 7% Ammonia solution up to pH 7.0

The neutralized mass volume was found to be 38 L and the Dimethyl acetamide content was 16%. It also contained Chlorinated sucrose derivatives and inorganic salts dissolved in it.

38 L of the said neutralized mass containing 16% of DMF and dissolved inorganic salts was passed through ADS 600 resin obtained from Thermax packed in SS column. The flow through from the column had DMF, inorganic salts and water and the 6-acetyl TGS was bound to the resin column. The column was then washed with water to remove any DMF and inorganics adhering to the resin. Then the flow through and washings collected was taken for DMF recovery. The total volume was 42 L containing 14% DMF.

Recovery of Dimethyl acetamide by affinity chromatography: The said flow through of collected flow through solution was passed through 60 L of HP20 resin obtained from Diaion (Mitsubishi Chemical Corporation, 33-8 Shiba 5-chome, Minato-ku, Tokyo 108-0014 Japan) packed in SS column. The solution was passed at a flow rate of 42 L/H. The flow through from the column had inorganic salts in water. The Dimethyl Acetamide was selectively adsorbed based on hydrophobic interaction chromatography to the resin. This flow through stream was collected and taken for waste management.

After the solution was passed, the column was washed with 120 L of DM water at 45 L/H. Then the adsorbed Dimethyl Acetamide in the resin was eluted with 15 L of methanol.

The Dimethyl Acetamide along with methanol was collected from the bottom of the column and was subjected to distillation at 45° C. under vacuum for methanol removal. The Dimethyl Acetamide obtained was checked for purity by GC and was found to be 96.2%. The overall yield of DMF from the recovery stream was 93%.

### Example 6

### Recovery of DMF from Roxithromycin Preparation

Generation of process stream: Erythromycin A oxime (37.5 g, 0.05 mole) is dissolved in dimethyl formamide (DMF) (100 ml) and cooled to 0-5.degree. C. Sodium methoxide (3.24 g, 0.062 mole) is added followed by (methoxyethoxy)methyl chloride (6.85 g, 0.055 mole) dissolved in DMF (12.5 ml), slowly with stirring, over 2-3 hours at 0-5.degree. C. The reaction is monitored by TLC until erythromycin A oxime disappears. Then the reaction mixture temperature is raised to ambient and, water (350 ml) added over 1 hour. The slurry is stirred for 2 hours, then the crystalline precipitate is collected by filtration and thoroughly washed with water (200 ml).

The filtrate was containing DMF up to 18% in water. This solution was subjected to DMF recovery using the HP20 resin from Diaion.

Recovery of DMF by affinity chromatography: This solution was passed through 100 ml of HP20 obtained from Diaion resin (resin details in Example 2) packed in SS column. The solution was passed at a flow rate of 100 ml/H. The flow through from the column was water and was sent directly to waste management. This stream was collected and taken for waste management. The DMF was selectively adsorbed to the resin. After the solution was passed, the column was washed with 250 ml of DM water at 100 ml/H. Then the adsorbed DMF in the resin was eluted with 100 ml of methanol.

The DMF along with methanol was collected from the bottom of the column and was subjected to distillation at 45° C. under reduced pressure for methanol removal. The DMF obtained was checked for purity by GC and was found to be 96.2%. The overall yield of DMF from the recovery stream was 98%.

1. A process of recovery and purification of a tertiary amide from an aqueous liquid composition, the said composition comprising a tertiary amide, one or more of an aqueous component and with or without one or more of an inorganic impurity, the said process comprising steps of:
a. contacting the said aqueous liquid composition with an adsorbent having a selective affinity towards the said tertiary amide,
b. washing the said adsorbent free from impurities by washing with an appropriate wash solvent that shall not desorb the adsorbed tertiary amide,
c. desorbing the adsorbed tertiary amide in a suitable solvent as an eluent and collecting it separately from the adsorbent,
d. separating the said eluent from the desorbed tertiary amide using a separation method and recovering the said tertiary amide in a substantially pure form.

2. A process of claim 1 wherein:
a. the said aqueous liquid composition is aqueous solution of a tertiary amide needing recovery of the said tertiary amide free from inorganic impurities including at least one aqueous constituent,
b. the said aqueous liquid composition is a process stream originating from one or more of a chemical process for manufacture of a product of an organic synthesis reaction,
c. the said tertiary amide comprises Dimethyl formamide, Dimethyl acetamide, N-methylpyrrolidine,
d. the said adsorbent is an aromatic type adsorbent based on crosslinked polystyrenic matrix coupled with an aromatic hydrophobic group preferably a benzene ring; preferably HP20 resin obtained from Diaion,
e. the said wash solvent comprises an aqueous solvent, preferably including water,
f. the said eluent comprises a polar alcoholic or organic solvent,
g. the said separation method comprises a distillation preferably under reduced pressure.

3. A process of claim 2 wherein the said chemical process comprises a process for preparation of 4,1′,6′ trichlorogalactosucrose (abbreviated as TGS) or TGS-6-ester.

4. A process of claim 3 comprising:
a. a process stream from chlorination of sucrose-6-ester, preferably of sucrose-6-acetate, optionally followed by deacetylation, resulting into a process stream comprising a DMF as preferred tertiary amide and one or more of a TGS-6-acetate, TGS, an organic impurity, an inorganic impurity and another constituent if added to the reaction mixture,
b. passing the process stream through a resin having selective affinity towards TGS-6-acetate or TGS, and other organic constituents except DMF, preferably ADS 600 resin obtained from Thermax, to adsorb the organic constituents except DMF and allow DMF and inorganic impurities unadsorbed to flow through,
c. washing the column by water to wash away DMF and inorganic impurities,
d. collecting the flow-through containing DMF, inorganic impurities and water aqueous process stream,
e. passing the said aqueous process stream through a column packed with a bed of preferred resin HP20, to get DMF selectively adsorbed on to the adsorbent and other constituents of the said process stream get washed away unadsorbed,
f. washing the said column with water to wash away unadsorbed residues of impurities/other constituents,
g. passing an eluent preferably comprising methanol; or alternatively one or more of acetone, acetonitrile, ethanol, isopropanol and the like; through the column to desorb and elute out the DMF,
h. isolating DMF from the eluted out DMF:eluent mixture by distilling out the eluent used, preferably methanol, under reduced pressure at 200 mmHg to atmospheric pressure of 760 mmHg leaving behind DMF at around 95% purity or more.

5. A process of claim 4 wherein the process stream of chlorination of sucrose6-acetate as preferred sucrose-6-ester results from a process comprising following steps:
a. preparing a Vilsmeier Reagent of general formula
i. [HClC═N⁺R₂]⁺Cl⁻ where R represents an alkyl group, typically a methyl or ethyl group, by one or more of a method of its preparation by reacting a tertiary amide, preferably DMF, with an acid chloride or [Bis(trichloromethyl)carbonate] (C₃O₃Cl₆) including a method of reacting DMF with an acid chloride comprising Phosphorus Pentachloride, thionyl chloride, phosgene and the like, or
ii. [HPOCl.₂.O.C⁺═N⁺.R.₂]Cl.⁻ where R represents an alkyl group, typically a methyl or ethyl group—by one or more of a method of its preparation by reacting a tertiary amide, preferably DMF, with phosphorus oxychloride,
b. adding sucrose-6-acetate solution, made preferably in DMF, to a Vilsmeier reagent of the step (a.) of this claim,
c. heating the reaction mass to around 85° C., and maintaining the same for a period of time, preferably for about 60 minutes,
d. then further heating to around 100° C., and maintaining the same for a period of time, preferably for about 5 hours, and
e. then further heating to around 115° C. and maintaining the same for a period of time, preferably for around 90 minutes,
f. cooling the chlorinated mass to lower temperature, preferably around 60° C.,
g. neutralizing the said cooled chlorinated mass with an alkali, preferably by calcium hydroxide slurry in water up to pH 7.0, optionally concentrating the same, thereafter, preferably by a non-evaporative concentration step including reverse osmosis,
h. submitting the process stream obtained at the end of the step (g.) of this claim for concentration under reduced pressure without further purification.

6. A process of claim 5 wherein the said process of manufacture comprising chlorination of sucrose-6-acetate as a preferred sucrose-6-ester further comprises one or more of a process including, as an illustration, chlorination by using thionyl chloride by:
a. taking DMF in a stirred glass lined reactor,
b. adding to it an anti-bumping agent, preferably charcoal,
c. providing Nitrogen sparging into the reaction mass,
d. adding thionyl chloride dropwise controlling the temperature between about 40 and 45° C. with constant stirring,
e. after completion of addition of thionyl chloride, stirring the mass at 45° C. for 60 minutes and then cooling to about 0-5° C.,
f. adding sucrose-6-acetate in DMF to the mass slowly controlling the temperature preferably to below about 5° C.,
g. then allowing the mass to come to ambient temperature and stirred preferably for about 3 hours,
h. heating the mass to about 85° C. and maintaining at that temperature preferably for 60 minutes,
i. further heating to about 100° C. and maintaining at that temperature for preferably 6 hours,
j. further heating to about 114° C. and maintaining for about 90 minutes,
k. then neutralizing to a preferable pH of around 7 using an alkali, preferably by using 7% ammonia solution and quenching the reaction resulting into a neutralized mass having DMF to about 15%, chlorinated sucrose derivatives, organic impurities and inorganic salts dissolved in it.
